(19) United States
(12) Patent Application Publication
(10) Pub. No.: US 2011/0086020 A1
(43) Pub. Date: Apr. 14, 2011

(54) Agent exhibiting immunoregulatory properties and the use thereof for treating autoimmune diseases

(76) Inventors: Khallar Abdumuslimovich Alikhanov, Orekhovy (RU); Leonid Yazonovich Kacharava, Mirtskulava (DE); Bagoadi Asumusumovich Alikhanov, Novokosinskaya (RU); Peter Georgievich Prokopenko, Novoyasenevsky (RU)

(21) Appl. No.: 12/798,027

(22) Filed: Mar. 29, 2010

(30) Foreign Application Priority Data
Apr. 28, 2006 (RU) (RU) 2006114457
Apr. 27, 2007 (RU) PCT/RU2007/000206

(51) Int. Cl. A61K 39/395; A61P 37/06

(52) U.S. Cl. 424/130.1

(57) ABSTRACT

The invention relates to medicine. The inventive agent which exhibits immunoregulatory properties and accelerated clinical performance at a recrudescence stage and is mainly used for treating autoimmune diseases comprises trophoblastic β-1-glycoprotein and immunoglobulin (Ig) which is embodied in the form of a multivalent ligand G (Ig-G) or A (Ig-A) or M (Ig-M) immunoglobulin. According to the second invention, an agent for treating autoimmune diseases comprises β-1-glycoprotein and immunoglobulin (Ig-G), wherein the multivalent ligand G or A (Ig-A) is used in the form of an immunoglobulin and the β-1-glycoprotein and immunoglobulin (Ig-G) are taken in equal portions on in portions whose ratio is equal to 1:19, respectively. For treating autoimmune diseases, the agent containing β-1-glycoprotein and immunoglobulin (Ig) is parenterally administratable.

### CROSS-REFERENCE TO RELATED APPLICATION

This application claims the benefit of the priority filing date of international application no. PCT/RU2007/000206, and Russian application no. (RU) 2006114457 filed on Apr. 28, 2006.

### FEDERALLY SPONSORED RESEARCH

Not Applicable

### SEQUENCE LISTING OR PROGRAM

Not Applicable

### STATEMENT REGARDING COPYRIGHTED MATERIAL

Portions of the disclosure of this patent document contain material that is subject to copyright protection. The copyright owner has no objection to the facsimile reproduction by anyone of the patent document or the patent disclosure as it appears in the Patent and Trademark Office file or records, but otherwise reserves all copyright rights whatsoever.

### BACKGROUND

The inventions pertain to the field of medicine, particularly to new biologically active substances (BAS) with immunoregulatory properties and to the use thereof. They can be used in practical medicine for treatment of autoimmune diseases, as well as in experimental biochemistry and veterinary medicine.

Immunoglobulins for intravenous administration used for the treatment of autoimmune diseases are known (see Pacetti P., Garau D., Caramatti C., Mangoni L., Zamboni V., Canova N., Rizzoli V. Assessment of the Efficacy of a Last-Generation Polyvalent Immunoglobulin In the Treatment of Idiopathic Thrombocytopenic Purpura//Curr. Med. Res. Opin.-1997.-13.-No. 9.-P. 517-527). However, the known agent is not intended for use at the acute stage of an autoimmune disease. It is only intended for use during the remission stage. In addition, to achieve clinical performance, it is necessary to infuse large doses of immunoglobulin continuously for several years.

As far as the technical background is concerned, the closest to the claimed inventions is trophoblastic β-1-glycoprotein (TBG) with immunoregulatory properties, intended for treatment of various autoimmune diseases (see RF patent No. 2056852, cl. A 61 K 35/50, 1994).

However, when used at the acute stage of an autoimmune disease, the effect of the known agent can only occur 4-5 days after its introduction into the patient's body.

The object of the invention is to develop an agent with immunoregulatory properties and to accelerate the clinical effect at the acute stage, mainly when treating autoimmune diseases.

### SUMMARY

The object of the invention is to develop an agent with immunoregulatory properties and to accelerate the clinical effect at the acute stage, mainly when treating autoimmune diseases.

To achieve this technical objective, an agent is proposed that can induce the suppressor activity and cytokine secretion of mononuclear cells. The agent contains trophoblastic β-1-glycoprotein (TBG) which, according to the invention, also contains immunoglobulin (Ig). In addition, class G (Ig-G), or class A (Ig-A), or class M (Ig-M) immunoglobul is used as the immunoglobulin. TBG and Ig-G are used in equal ratio or in the ratio of 1:19, respectively.

According to the second invention, for the treatment of autoimmune diseases one uses an agent with the property to induce the suppressor activity and cytokine secretion of mononuclear cells. The agent comprises TBG and Ig, wherein class G (Ig-G), or class A (Ig-A), or class M (Ig-M) immunoglobulin is used as the immunoglobulin. TBG and Ig-G are used in equal ratios or in the ratio of 1:19, respectively. In addition, when treating autoimmune diseases the agent comprising TBG and Ig is administered parenterally.

The essence of the invention is that the claimed agent that comprises TBG and Ig has the property to suppress the proliferative activity of mononuclear cells, and to induce the suppressor activity and secretion of cytokines TFR-V1, IL-10, IL-6. In addition, the use of the claimed agent makes it possible to achieve a stronger clinical effect that occurs during the 1^{st} day after the agent is administered.

### DESCRIPTION

To achieve this technical objective, an agent is proposed that can induce the suppressor activity and cytokine secretion of mononuclear cells. The agent contains trophoblastic β-1-glycoprotein (TBG) which, according to the invention, also contains immunoglobulin (Ig). In addition, class G (Ig-G), or class A (Ig-A), or class M (Ig-M) immunoglobul is used as the immunoglobulin. TBG and Ig-G are used in equal ratio or in the ratio of 1:19, respectively.

According to the second invention, for the treatment of autoimmune diseases one uses an agent with the property to induce the suppressor activity and cytokine secretion of mononuclear cells. The agent comprises TBG and Ig, wherein class G (Ig-G), or class A (Ig-A), or class M (Ig-M) immunoglobulin is used as the immunoglobulin. TBG and Ig-G are used in equal ratios or in the ratio of 1:19, respectively. In addition, when treating autoimmune diseases the agent comprising TBG and Ig is administered parenterally.

The essence of the invention is that the claimed agent that comprises TBG and Ig has the property to suppress the proliferative activity of mononuclear cells, and to induce the suppressor activity and secretion of cytokines TFR-V1, IL-10, IL-6. In addition, the use of the claimed agent makes it possible to achieve a stronger clinical effect that occurs during the 1^{st} day after the agent is administered.

TBG can be manufactured from the production waste of gamma globulin (fraction A) (certificate of authorship No. 1341736, cl. A 61 K35/16, 1985).

Immunoglobulin Ig can be manufactured using a commonly known method (see, for instance, V. V. Anastasiev, “Immunoglobulin For Intravenous Administration”, Nizhniy Novgorod, NGMA, 2000).

The claimed agent can be manufactured by mixing initial components, TBG and Ig, in a glass dish at +1÷10° C. in the ratios of 1:99, 1:19, 1:9, 1:1.5, and 1:1.

The curative effect of the claimed agent is that it has the property to induce the suppressor activity of mononuclear cells of patients with autoimmune diseases and secretion of TFR-V1 and IL-10 cytokines by mononuclear cells.

This makes it possible to claim that the claimed agent has the immunoregulatory property.

It should be noted that the agent was made from the above components, TBG and Ig-G, in various ratios listed above. However, no significant differences were noticed when making the agent with different ratios of the components.

However, taking into account an economic approach, the TBG and Ig-G ratio of 1:19 is recommended.

This ratio is optimal, both from the standpoint of the effective action of the agent and from the standpoint of convenience of making it, although during research a positive result was also obtained repeatedly at the ratio of 1:99.

It should be noted that various Ig classes (types) were used—Ig-G, Ig-A [symbol] Ig-M, and approximately identical results were produced. However, in practice, the Ig-G class is preferred because manufacturing it is simpler.

### PREFERRED EMBODIMENT OF INVENTION

The biological activity of the claimed agent comprising TBG and Ig was determined as follows.

### Example 1

The action of an agent comprising TBG and Ig-G was studied for the proliferative activity of mononuclear cells of peripheral blood that were extracted using the 1969 Boyum method.

The range of used doses was 1 mcg/ml-960 mcg/ml.

The study results are shown in Table 1.

TABLE 1

| TBG (Pure) | 1 | 3 | 6 | 15 | 30 | 60 | 120 | 240 | 480 | 960 |
|---|---|---|---|---|---|---|---|---|---|---|
| Dose, mcg/ml | | | | | | | | | | |
| Inhibition | 0 | 4 | 14 | 23 | 31 | 44 | 37 | 23 | 13 | 0 |
| Index, % | | | | | | | | | | |
| TBG Dose In | 0.5 | 1.5 | 3 | 7.5 | 15 | 30 | 60 | 120 | 240 | 480 |
| the Agent | | | | | | | | | | |
| With Ig-G | | | | | | | | | | |
| Inhibition | 0 | 5 | 15 | 24 | 32 | 46 | 38 | 25 | 14 | 0 |
| Index, % | | | | | | | | | | |

One can see from Table 1 that the agent comprising TBG and Ig-G suppresses the proliferative activity of mononuclear cells of peripheral blood caused by phytohemagglutinin twice as strongly as pure TBG.

### Example 2

The effect of the claimed agent comprising TBG and Ig-G on the ability to induce the suppressor activity of mononuclear cells of peripheral blood was studied. During the first stage, an MNS suspension is produced using the method of cell sedimentation in a one-step ficoll-urografin gradient (the Boyum method).

Peripheral blood is drawn from the donor by venipuncture, always at the same time, and placed in vials with heparin solution based on 1 ml of blood per 20-30 units of heparin. Then blood is diluted with Hinks solution with no Ca⁺⁺ and Mg⁺⁺ in the ratio of 1:2 and layered on the ficoll-urografin gradient (density—1.078).

Then centrifuging is performed for 30 minutes in a 400 g mode. The MNC suspension is transferred from the interphase into a centrifuge vial, Hanks solution with no Ca⁺⁺ and Mg⁺⁺ is added, and three successive 10-minute centrifuging are performed to wash the cells from the ficoll-urografin solution. After the third centrifuging, the MNC sediment is re-suspended in 1 ml of 199 medium, and the number of mononuclear cells is calculated using a Goryaev chamber.

During the second stage the MNC are split into two equal parts. The first part is cultured without the suppressor activator, and the second part is cultured with a suppressor activator, the studied agent being used as the suppressor activator.

The MNC are cultured in penicillin vials plugged with rubber plugs No. 14.5 at 37° C. The culture medium is RPMI-1640 with 20% of serum of the IV AB blood group serum and glutamine added.

In each vial, 5×10⁶ cells are cultured in 2.0 ml of full medium. The agent comprising TBG and Ig-G in 1-960 mcg/ml doses is added to the culture to induce suppressors.

The cells are cultured for 48 hours. Then the MNC are washed off the culture medium, and proliferation is blocked by treatment with mitomycin C—40 mcg/ml for 30 minutes at 37° C. Then they are washed three times with 199 medium with 5% of (cooled) IV AB serum. The cell sediment is re-suspended. The number of nuclear-containing cells is calculated. The percentage of viable cells is determined using 0.1% blue tripsin solution, and it is diluted to the required concentration. In this case, all operations are performed separately with control cells and stimulated composition. To wash the cells, siliconized dishes are used.

At the next stage, freshly extracted MNC, stimulated by phitohemaagglutamin (FGA) and that play the role of responding test cells, are added to each part of control and stimulated MNC in equal ratios (0.5×10⁶ cell/ml) to obtain test cultures. They are cultured for 72 hours. Then, proliferation of test cultures is evaluated using H³-thymidine, and the amount of suppression is judged by the degree of proliferation decrease in them. The suppression index (SI) is determined from the following formula:

$$SI = ( 1 - \frac{\begin{matrix}number of pulse / min in the test \\ culture stimulated by the agent\end{matrix}}{\begin{matrix}number of pulse / min in the test \\ culture without the agent\end{matrix}} ) 100 \% .$$

The results of studies of the effect of the agent comprising TBG and Ig-G on the ability to induce the MNC suppressor activity are shown in Table 2. The dose range is 1-960 mcg/ml.

TABLE 2

| TBG (Pure) | 1 | 3 | 6 | 15 | 30 | 60 | 120 | 240 | 480 | 960 |
|---|---|---|---|---|---|---|---|---|---|---|
| Dose, mcg/ml | | | | | | | | | | |
| Suppression | 0 | 2 | 7 | 16 | 23 | 40 | 26 | 13 | 4 | 0 |
| Index, % | | | | | | | | | | |
| TBG Dose In | 0.5 | 1.5 | 3 | 7.5 | 15 | 30 | 60 | 120 | 240 | 480 |
| the Agent | | | | | | | | | | |
| With Ig-G | | | | | | | | | | |
| Suppression | 0 | 3 | 8 | 17 | 24 | 44 | 27 | 15 | 6 | 0 |
| Index, % | | | | | | | | | | |

One can see from Table 2 that the agent comprising TBG and Ig-G suppresses the proliferative activity of mononuclear cells of peripheral blood twice as strongly as pure TBG.

### Example 3

The effect of the agent comprising TBG and Ig-G on the ability to induce production of cytokines—transforming growth factor beta 1 (TGF-β1) and interleukine-10 (IL-10) by mononuclear cells (MNC) of peripheral blood was studied. During the first stage, an MNC suspension was obtained using the method of cell sedimentation in a one-step ficoll-urografin gradient (the Boyum method).

The MNC were split into two equal parts; the first part was cultured without the claimed agent, and the second part with the claimed agent.

The MNC are cultured in penicillin vials plugged with rubber plugs No. 14.5 at 37° C. The culture medium is RPMI-1640 with 20% of serum of the IV AB blood group serum and glutamine added.

In each vial, 5×10⁶ MNC were cultured in 1 ml of full medium. The claimed agent was added to the MNC culture to induce production of cytokines TFR-β1 and IL-10.

The cells were cultured for 24 hours. Then, the culture medium was separated from the cells by centrifuging (1000 g, 10 minutes), and 500 mcl of it was taken for analysis.

When determining the amount of TFR-β1 in the culture medium, specimens were first extracted. This stage of the analysis makes it possible to free TFR-β1 from complexes, making it traceable.

To do this, 0.25 ml (250 mcl) of culture medium and 0.05 ml (50 mcl) of extracting solution was placed in a polyethylene vial. The contents were mixed in a vibration mixer and incubated for 30 minutes at 4° C. 250 mcl of process buffer solution for standards dilution was added to the vial. At this stage, the culture medium is diluted 1:2.2.

Then, the number of 8-well strips required for analysis were taken from a folding microplate covered with antibodies to TFR-β1.

To increase the validity of the analysis results, the studied and control specimens were placed in duplicates, using two wells for each specimen. 200 mcl of each standard and 200 mcl of the extracted specimen or clotrone were placed in respective wells. 500 mcl of biotin-conjugated antibodies to TFR-β1 were added to each well and mixed by tapping the plate. The plate was covered with film, and incubated for 30 minutes at room temperature. The well contents were completely removed. All wells were washed 4 times with process buffer solution. Each time, 400 ml of the solution was added to each well, and each time the solution was poured in and removed from the strip wells. After the washing was finished, the remaining moisture was removed by tapping the turned-upside-down strips on filter paper. 100 mcl of process solution of streptavidin peroxidase conjugate was added to each well, except to the chromogen blank. The plate was covered with film, and incubated for 30 minutes at room temperature. The well contents were completely removed. All wells were washed 4 times with process buffer solution. Then, 100 mcl of chromogen solution of tetramethyl benzdine (TMB) was added to each well. They were incubated for 20-30 minutes in a dark at room temperature until blue staining appeared in wells with standards having a maximum content of TFR-β1.

100 mcl of stop solution (1H sulfuric acid solution) was added to each well to stop the enzyme reaction. The reagents were mixed by gingerly tapping the strip holder. The solution gradually changed its color from blue to yellow.

The results were recorded photometrically using a photometer for immune-enzyme analysis at the 450 nm wavelength immediately after the enzyme reaction was stopped.

To determine the concentration of TFR-β1 in the studied specimens, a calibration curve was plotted: X-axis—the concentration of TFR-β1 in the standards (rg/ml); Y-axis—the respective value of optical density.

The concentration of TFR-β1 in the studied specimens was determined from the calibration curve based on the obtained values of optical density. If the specimens were diluted, the derived concentration values were multiplied by the dilution coefficient (10, 100, 1000, etc.).

To determine the amount of IL-10 in the culture medium, 8-well strips (required number for analysis) were taken from a folding microplate covered with antibodies to IL-10.

To increase the validity of the analysis results, the studied and control specimens were placed in duplicates, using two wells for each specimen.

50 mcl of each standard, tested specimen or control specimen were placed in respective wells. 50 mcl of incubation buffer were added to the wells with standards and 50 mcl of diluting process solution were added to the wells with the culture medium.

The plate was covered with film and incubated for 2 hours at room temperature.

The well contents was completely removed.

All wells were washed 4 times with process buffer solution. Each time, 400 ml of the solution was added to each well, and each time the solution was poured in and removed from the strip wells. 100 mcl of biotine-conjugated antibodies to IL-10 were added to each well and mixed by tapping the plate.

The plate was covered with film and incubated for 2 hours at room temperature. The well contents were completely removed. All wells were washed 4 times with process buffer solution. 100 mcl of process solution of streptavidin peroxidase conjugate was added to each well, except to the chromogen blank.

The plate was covered with film and incubated for 30 minutes at room temperature. The well contents were completely removed. The wells were washed 4 times with process buffer solution.

Then, 100 mcl of chromogen solution of tetramethyl benzdine (TMB) was added to each well.

They were incubated for 20-30 minutes in the dark at room temperature until blue staining appeared in wells with standards having a maximum content IL-10.

100 mcl of stop solution (1H sulfuric acid solution) was added to each well to stop the enzyme reaction. The reagents were mixed by gingerly tapping the strip holder. The solution gradually changed its color from blue to yellow.

The results were also recorded photometrically using a photometer for immune-enzyme analysis at the 450 nm wavelength immediately after the enzyme reaction was stopped.

To determine IL-10 in the studied specimens, a calibration curve was plotted: X-axis—the concentration of IL-10 in the standards (rg/ml); Y-axis—the respective value of optical density.

The concentration of IL-10 in the studied specimens was determined from the calibration curve based on the obtained values of optical density. If the specimens were diluted, the derived concentration values must be multiplied by the dilution coefficient (10, 100, 1000, etc.).

The result of the studies showed that an agent comprising TBG and Ig-G has the ability to induce simultaneous production of two cytokines, TFR-β1 and IL-10, by mononuclear cells of peripheral blood.

And the level of IL-10 and TFR-β1 cytokines, produced by mononuclear cells of peripheral blood when exposed to the claimed agent, is 2-3 times higher than when exposed to pure TBG. Moreover, when administered to a patient, the claimed agent alleviates exacerbation of autoimmune diseases. Its action is pronounced during the very first day of treatment, whereas when pure TBG is administered to a patient, the action is only pronounced during the fourth or fifth day of treatment.

### Example 4

The effect of the agent comprising TBG and Ig-G in the TBG-Ig-G ratio of 1:99 on the proliferative activity of mononuclear cells of peripheral blood extracted using the 1969 Boyum method was studied.

The range of the used doses was 1 mcg/ml-960 mcg/ml.

The study results are shown in Table 3.

TABLE 3

| | | | | | | | | | | |
|---|---|---|---|---|---|---|---|---|---|---|
| TBG (Pure) | 1 | 3 | 6 | 15 | 30 | 60 | 120 | 240 | 480 | 960 |
| Dose, mcg/ml | | | | | | | | | | |
| Inhibition | 0 | 4 | 14 | 23 | 31 | 44 | 37 | 23 | 13 | 0 |
| Index, % | | | | | | | | | | |
| TBG Dose In | 0.5 | 1.5 | 3 | 7.5 | 15 | 30 | 60 | 120 | 240 | 480 |
| the Agent | | | | | | | | | | |
| With Ig-G | | | | | | | | | | |
| Inhibition | 0 | 3 | 13 | 24 | 33 | 45 | 37 | 24 | 14 | 0 |
| Index, % | | | | | | | | | | |

One can see from Table 3 that the agent comprising TBG and Ig-G in the ratio of 1:99 suppresses the proliferative activity of mononuclear cells of peripheral blood caused by phytohemagglutinin at the same extent as the claimed agent comprising TBG and Ig-G in the ratio of 1:19.

### Example 5

The effect of the agent comprising TBG and Ig-G in the TBG-Ig-G ratio of 1:1.5 on the proliferative activity of mononuclear cells of peripheral blood extracted using the 1969 Boyum method was studied.

The range of the used doses was 1 mcg/ml-960 mcg/ml.

The study results are shown in Table 4.

TABLE 4

| | | | | | | | | | | |
|---|---|---|---|---|---|---|---|---|---|---|
| TBG (Pure) | 1 | 3 | 6 | 15 | 30 | 60 | 120 | 240 | 480 | 960 |
| Dose, mcg/ml | | | | | | | | | | |
| Inhibition | 0 | 4 | 14 | 23 | 31 | 44 | 37 | 23 | 13 | 0 |
| Index, % | | | | | | | | | | |
| TBG Dose In | 0.5 | 1.5 | 3 | 7.5 | 15 | 30 | 60 | 120 | 240 | 480 |
| the Agent | | | | | | | | | | |
| With Ig-G | | | | | | | | | | |
| Inhibition | 0 | 4.5 | 13 | 22 | 33 | 45 | 37.5 | 24 | 15 | 0 |
| Index, % | | | | | | | | | | |

One can see from Table 4 that the agent comprising TBG and Ig-G in the ratio of 1:1.5 suppresses the proliferative activity of mononuclear cells of peripheral blood caused by phytohemagglutinin at the same extent as the claimed agent comprising TBG and Ig-G in the ratio of 1:19 and 1:99.

### Example 6

The effect of the agent comprising TBG and Ig-G in the TBG-Ig-G ratio of 1:9 on the proliferative activity of mononuclear cells of peripheral blood extracted using the 1969 Boyum method was studied.

The range of the used doses was 1 mcg/ml-960 mcg/ml.

The study results are shown in Table 5.

TABLE 5

| | | | | | | | | | | |
|---|---|---|---|---|---|---|---|---|---|---|
| TBG (Pure) | 1 | 3 | 6 | 15 | 30 | 60 | 120 | 240 | 480 | 960 |
| Dose, mcg/ml | | | | | | | | | | |
| Inhibition | 0 | 4 | 14 | 23 | 31 | 44 | 37 | 23 | 13 | 0 |
| Index, % | | | | | | | | | | |
| TBG Dose In | 0.5 | 1.5 | 3 | 7.5 | 15 | 30 | 60 | 120 | 240 | 480 |
| the Agent | | | | | | | | | | |
| With Ig-G | | | | | | | | | | |
| Inhibition | 0 | 3.5 | 13 | 22 | 29 | 42 | 36 | 26 | 14 | 0 |
| Index, % | | | | | | | | | | |

One can see from Table 5 that the agent comprising TBG and Ig-G in the ratio of 1:9 suppresses the proliferative activity of mononuclear cells of peripheral blood caused by phytohemagglutinin at the same extent as the claimed agent comprising TBG and Ig-G in the ratio of 1:19, 1:99 and 1:1.5.

### Example 7

The effect of the agent comprising TBG and Ig-G in the TBG-Ig-G ratio of 1:1 on the proliferative activity of mononuclear cells of peripheral blood extracted using the 1969 Boyum method was studied.

The range of the used doses was 1 mcg/ml-960 mcg/ml.

The study results are shown in Table 6.

TABLE 6

| | | | | | | | | | | |
|---|---|---|---|---|---|---|---|---|---|---|
| TBG (Pure) | 1 | 3 | 6 | 15 | 30 | 60 | 120 | 240 | 480 | 960 |
| Dose, mcg/ml | | | | | | | | | | |
| Inhibition | 0 | 4 | 14 | 23 | 31 | 44 | 37 | 23 | 13 | 0 |
| Index, % | | | | | | | | | | |
| TBG Dose In | 0.5 | 1.5 | 3 | 7.5 | 15 | 30 | 60 | 120 | 240 | 480 |
| the Agent | | | | | | | | | | |
| With Ig-G | | | | | | | | | | |
| Inhibition | 0 | 5 | 11 | 24 | 29 | 45 | 36 | 22 | 14 | 0 |
| Index, % | | | | | | | | | | |

One can see from Table 6, that the agent comprising TBG and Ig-G in the ratio of 1:9 suppresses the proliferative activity of mononuclear cells of peripheral blood caused by phytohemagglutinin at the same extent as the claimed agent comprising TBG and Ig-G in the ratio of 1:19, 1:99, 1:9 and 1:1.5.

### Example 8

The results of studies of the effect of the agent comprising TBG and Ig-G in the ratio of 1:99 on the ability to induce the MNC suppressor activity are shown in Table 7. The dose range is 1-960 mcg/ml.

TABLE 7

| | | | | | | | | | | |
|---|---|---|---|---|---|---|---|---|---|---|
| TBG (Pure) | 1 | 3 | 6 | 15 | 30 | 60 | 120 | 240 | 480 | 960 |
| Dose, mcg/ml | | | | | | | | | | |
| Suppression | 0 | 2 | 7 | 16 | 23 | 40 | 26 | 13 | 4 | 0 |
| Index, % | | | | | | | | | | |
| TBG Dose In | 0.5 | 1.5 | 3 | 7.5 | 15 | 30 | 60 | 120 | 240 | 480 |
| the Agent | | | | | | | | | | |
| With Ig-G | | | | | | | | | | |
| Suppression | 0 | 3.5 | 8 | 18 | 24 | 38 | 28 | 17 | 6 | 0 |
| Index, % | | | | | | | | | | |

One can see from Table 7 that the agent comprising TBG and Ig-G in the ratio of 1:99 induces the suppressor activity of mononuclear cells of peripheral blood at the same extent as the agent comprising TBG and Ig-G in the ratio of 1:19, and induces production of cytokines—transforming growth factor beta 1 TGF-β1 and interleukine-10 (IL-10) by mononuclear cells (MNC) of peripheral blood.

### Example 9

The results of studies of the effect of the agent comprising TBG and Ig-G in the ratio of 1:9 on the ability to induce the MNC suppressor activity are shown in Table 8.

The dose range is 1-960 mcg/ml.

TABLE 8

| | | | | | | | | | | |
|---|---|---|---|---|---|---|---|---|---|---|
| TBG (Pure) | 1 | 3 | 6 | 15 | 30 | 60 | 120 | 240 | 480 | 960 |
| Dose, mcg/ml | | | | | | | | | | |
| Suppression | 0 | 2 | 7 | 16 | 23 | 40 | 26 | 13 | 4 | 0 |
| Index, % | | | | | | | | | | |
| TBG Dose In | 0.5 | 1.5 | 3 | 7.5 | 15 | 30 | 60 | 120 | 240 | 480 |
| the Agent | | | | | | | | | | |
| With Ig-G | | | | | | | | | | |
| Suppression | 0 | 5 | 15 | 25 | 33 | 46 | 35 | 22 | 14 | 0 |
| Index, % | | | | | | | | | | |

One can see from Table 8 that the agent comprising TBG and Ig-G in the ratio of 1:9 induces the suppressor activity of mononuclear cells of peripheral blood at the same extent as the agent comprising TBG and Ig-G in the ratio of 1:19 and 1:99, and induces production of cytokines, transforming growth factor beta 1 TGF-β1 and interleukine-10 (IL-10), by mononuclear cells (MNC) of peripheral blood.

### Example 10

The results of studies of the effect of the agent comprising TBG and Ig-G in the ratio of 1:1.5 on the ability to induce the MNC suppressor activity are shown in Table 9.

The dose range is 1-960 mcg/ml.

TABLE 9

| | | | | | | | | | | |
|---|---|---|---|---|---|---|---|---|---|---|
| TBG (Pure) | 1 | 3 | 6 | 15 | 30 | 60 | 120 | 240 | 480 | 960 |
| Dose, mcg/ml | | | | | | | | | | |
| Suppression | 0 | 2 | 7 | 16 | 23 | 40 | 26 | 13 | 4 | 0 |
| Index, % | | | | | | | | | | |
| TBG Dose In | 0.5 | 1.5 | 3 | 7.5 | 15 | 30 | 60 | 120 | 240 | 480 |
| the Agent | | | | | | | | | | |
| With Ig-G | | | | | | | | | | |
| Suppression | 0 | 3.5 | 11 | 25 | 33 | 47 | 35 | 21 | 15 | 0 |
| Index, % | | | | | | | | | | |

One can see from Table 9 that the agent comprising TBG and Ig-G in the ratio of 1:1.5 induces the suppressor activity of mononuclear cells of peripheral blood at the same extent as the agent comprising TBG and Ig-G in the ratio of 1:19, 1:99 and 1:9, and induces production of cytokines, the transforming growth factor beta 1 TGF-β1 and interleukine-10 (IL-10), by mononuclear cells (MNC) of peripheral blood.

### Example 11

The results of studies of the effect of the agent comprising TBG and Ig-G in the ratio of 1:1 on the ability to induce the MNC suppressor activity are shown in Table 10.

The dose range is 1-960 mcg/ml.

TABLE 10

| | | | | | | | | | | |
|---|---|---|---|---|---|---|---|---|---|---|
| TBG (Pure) | 1 | 3 | 6 | 15 | 30 | 60 | 120 | 240 | 480 | 960 |
| Dose, mcg/ml | | | | | | | | | | |
| Suppression | 0 | 2 | 7 | 16 | 23 | 40 | 26 | 13 | 4 | 0 |
| Index, % | | | | | | | | | | |
| TBG Dose In | 0.5 | 1.5 | 3 | 7.5 | 15 | 30 | 60 | 120 | 240 | 480 |
| the Agent | | | | | | | | | | |
| With Ig-G | | | | | | | | | | |
| Suppression | 0 | 4 | 9 | 22 | 29 | 42 | 33 | 19 | 8 | 0 |
| Index, % | | | | | | | | | | |

One can see from Table 10 that the agent comprising TBG and Ig-G in the ratio of 1:1 induces the suppressor activity of mononuclear cells of peripheral blood at the same extent as the agent comprising TBG and Ig-G in the ratio of 1:19, 1:99, 1:9 and 1:1.5, and induces production of cytokines, the transforming growth factor beta 1 TGF-β1 and interleukine-10 (IL-10), by mononuclear cells (MNC) of peripheral blood.

### Example 12

### Case Record

Parient O., 43 years old, was undergoing medical treatment at the 5^{th} medical ward, with the diagnosis of seropositive rheumatoid arthritis-polyarthritis with systemic presentations (fever, lymphoadenopathy, anemia, amyotrophies, neuropathy, rheumatoid nodules), activity 111 st., stage 111, joint function deficiency 111.

At admission, Patient O complained about pain in the small and large joints of the extremities (proximal digital, metacarpophalngeal, radiocarpal, elbow, shoulder, knee, ankle, metatarsophalangeal, and sternoclavicular), joint swelling, restriction of joint movement, marked joint stiffness lasting all day long, tingling sensation in toe and finger tips, and temperature rise to 38.6°.

The patient noted arthralgias for 7 years and moderate joint pain with swelling for 5 years. The patient had been taking non-steroid anti-inflammatory preparations irregularly. Quite often the patient had used alcohol to alleviate pain. Current worsening had begun two weeks before hospitalization and after acute respiratory infection. The temperature had risen to febrile level, multiple joint swelling had developed, and the above listed complaints had appeared. Antibiotics and non-steroid anti-inflammatory preparations had been administered on an outpatient basis. The patient had recently started using alcohol, and has been hospitalized.

The patient's condition was poor at admission. The patient's body temperature was 38.6°, and was totally immobilized due to joint pain and swelling. The patient groans from the pain. The patient's skin teguments were pale. The patient had leg sponginess. Increased (1.5×1.5 cm, 1.5×2 cm) lymph nodes (mandibular, axillary, and parietal) were palpable. The patient had a rough time breathing in the lungs due to smoking. Heart tones were moderately muffled, tachycardia was up to 92 per minute, rhythmic pulse, satisfactory filling, and blood pressure 130/80 mm Hg. The abdomen was soft and painless. The liver was +2 cm. The spleen was not palpable. The Pasternatsky's symptom was negative.

The patient exhibited marked amyotrophias of the extremities. The patient also exhibited marked swelling and joint defiguration joints (proximal digital, metacarpophalngeal, radiocarpal, elbow, shoulder, knee, and ankle), and skin hyperthermia over the joints. The patient's joints were limited in active and passive movements. The patient exhibited Ulnar deviation of the hands, contractures of radiocarpal and elbow joints, and a rheumatoid nodule in the left elbow joint.

X-rays of the hands revealed osteoporosis, constriction of interarticular fissures, multiple erosions, and subluxations.

Blood tests revealed hemoglobin 86 g/l, W.B.C. 11,200, ESR 67 mm/h, CRP 4+, RF 1:320, HBs (−), AST 74, ALT 56, whole blood protein 66 g/l.

The suppressor activity of T-lymphosites of peripheral blood in the case of the claimed agent induction was 16%.

The patient's EKG revealed sinus rhythm and diffuse myocardial changes. Chest X-rays revealed no focal or infiltrative changes.

The patient was treated as follows: rheopirin—3 ml intramuscularly daily, diclofenac—50 mg 3 times a day, analgin with Benadryl injections—twice a day.

The Patient's condition showed no significant dynamics for 10 days, Anemia, fever up to 37.9-38.8°, and marked exudative changes in the joints persisted. The patient was immobilized.

The patient was administered a course of daily intravenous infusions with the claimed agent comprising TBG and Ig-G in the ratio of 1:99.

The patient's condition improved considerably. Joint pain and swelling reduced noticeably, and the scope of movements increased. Body temperature had normalized. Joint stiffness disappeared. The patient began walking and was able to take care of himself. Blood hemoglobin level increased to 125 g/l, ESR decreased to 27 mm/h, SRP dropped to 1+, and total blood protein increased to 84 g/l.

Reanalysis discovered the increase of the suppressor activity of T-lumphocites of peripheral blood in the case of the claimed agent induction—64.2%. In Ouchterlony's reaction on days 7, 14 and 28, no antibodies to the claimed agent were found.

The patient was discharged in satisfactory condition.

It was recommended that the patient continue taking non-steroid anti-inflammatory preparations (diclofenac) on an outpatient basis. It was also recommended to prescribe basic preparations (sulfasalazone).

### Example 13

Patient M., 35 years old, was undergoing medical treatment with the diagnosis of seropositive rheumatoid arthritis-polyarthritis with systemic presentations (amyotrophy, anemia, neuropathy, fever), activity II st., stage II, impaired joint function II.

At admission, he complained about joint pain (proximal digital, metacarpophalngeal, radiocarpal, elbow, shoulder, knee, ankle), swelling and limited mobility of those joints. He complained about marked joint stiffness from morning to lunch time, fingertip numbness, and body temperature increase to subfebrile levels.

He considered himself to be sick for 5 years. The illness started with affection of ankle and knee joints. In the last two years, swelling of the radiocarpal and interphalangeal joints appeared. Since that time, he began noticing joint stiffness in the morning. Treatment with non-steroid anti-inflammatory preparations was administered, with positive effect. Current worsening began 3 week ago, after an acute respiratory disease—marked swelling of the joints (proximal interphlangeal, metacarpophalangeal, knee, and ankle) developed, morning stiffness increased (and was continuing to lunch time), and temperature increased to 37.5°. Treatment with non-steroid anti-inflammatory preparations and antibiotics (klacid) had no significant effect, so he was hospitalized.

He was immobilized at admission. Walking was difficult and he walked with a cane.

The patient exhibited defiguration, swelling of the proximal interphalangeal, metacarpophalngeal, radiocarpal, knee, and ankle joints, and restricted active and passive joint movements. He also exhibited hyperthermia of the knee, radiocarpal and ankle joints, contractures of radiocarpal joints, and marked amyotrophias of the thigh and shoulder muscles. There was no marked organ pathology.

Blood tests revealed HB 110 g/l, W.B.C. 10700, ESR 45 mm/h, CRP 3+, RF 1:160, total protein 70 g/l, AST 40, ALT 34. Urinalysis showed density 1025, W.B.C. 1-2 within the field of view.

Hand joints X-rays revealed osteoporosis, constriction of interarticular fissures, and multiple erosions.

The patient's treatment was the following: non-steroid anti-inflammatory preparations (diclofenac injections, rheopirin injections), magnetic-laser therapy, and physical therapy.

His condition was without a marked positive dynamics. Subfebrility and joint swelling persisted, and joint stiffness increased. ESR increased to 48 mm/h, CRP 3+.

The suppressor activity of T-lymphocites of peripheral blood in the case of induction of the claimed agent comprising TBG and Ig-G was 20%.

Daily injections of the claimed agent comprising TBG and Ig-G (1:19) were prescribed.

The patient's condition improved considerably. Joint stiffness disappeared, and body temperature normalized. Joint pain and swelling decreased considerably, and the scope of joint motion increased. The patient began to walk easily within the ward. He was active. His hand grip strength increased from 5 mm HG to 90 mm Hg (left hand) and 110 mm Hg (right hand). Swelling of the ankle and knee joints disappeared completely. Blood hemoglobin level increased to 140 g/l. ESR dropped to 22 mm/h, and CRP became 1+.

Reanalysis discovered an increase of the suppressor activity of T-lumphocites of peripheral blood in the case of the claimed agent induction—60.4%. In Ouchterlony's reaction on days 7, 14 and 28, no antibodies to the claimed agent were found.

The patient was discharged in satisfactory condition for outpatient treatment.

To study the safety of the claimed agent comprising TBG and Ig-G, its acute toxicity was studied prior to clinical use. Acute toxicity was studied in accordance with the Pharmacological Committee of the Russian Federation Guidelines “Requirements to Pre-Clinical Studies of General Toxic Action of New Pharmacological Substances”, Moscow, 2001. The study results demonstrated that intraperitoneal introduction of a thousand-fold dose of the claimed agent had no acute toxic action, and without these doses it turned out to be impossible to achieve its LD₅₀. Thus, the proposed agent comprising TBG and Ig-G is not toxic.

Although the above inventions have been described in considerable detail, it will be clear to an average skilled professional in the field that, in light of the description of these inventions, certain changes and modifications of the inventions can be made without digressing from the idea or scope of the proposed formulas of the inventions.

The outlined advantages of the proposed agent comprising TBG and Ig offer the prospect of widespread application both in scientific research and in practical medicine, veterinary and experimental biochemistry.

The invention claimed is:

1. An agent with the property to induce the suppressor activity and cytokine secretion of mononuclear cells that contains trophoblastic β-1-glycoprotein (TBG), distinctive in that it also contains immunoglobulin (Ig).

2. An agent per item 1, distinctive in that immunoglobulin class G (Ig-G), or class A (Ig-A), or class M (Ig-M) is used as immunoglobulin.

3. An agent per item 1, distinctive in that TBG and Ig are used in equal ratio.

4. An agent per item 1, distinctive in that TBG and Ig are used in the ratio of 1:19, respectively.

5. The use of an agent with the property to induce the suppressor activity and cytokine secretion of mononuclear cells that contains TBG and immunoglobulin (Ig) for treatment of autoimmune diseases.

6. The use of the agent per item 5, distinctive in that class G (Ig-G), or class A (Ig-A), or class M (Ig-M) immunoglobulin is used as immunoglobulin.

7. The use of an agent per item 5, distinctive in that it is administered parenterally.

8. The use of the agent per item 6, distinctive in that TBG and Ig are used in equal ratio.

9. The use of the agent per item 6, distinctive in that TBG and Ig are used in the ratio of 1:19, respectively.
